(19) United States
(12) Patent Application Publication
(10) Pub. No.: US 2024/0300164 A1
(43) Pub. Date: Sep. 12, 2024

(54) A METHOD

(71) Applicants: Imperial College Innovations Limited, London (GB); Ecole Polytechnique Federale de Lausanne, Lausanne (CH)

(72) Inventors: Guang-Zhong Yang, London (GB); Mohamed Ek Abdelaziz, London (GB); Burak Temelkuran, London (GB); Andreas Leber, Lausanne (CH); Fabien Sorin, Lausanne (CH)

(21) Appl. No.: 18/269,114

(22) PCT Filed: Dec. 22, 2021
(86) PCT No.: PCT/GB2021/053411

§ 371 Date: Jun. 22, 2023

(30) Foreign Application Priority Data
Dec. 22, 2020 (GB) 2020346.9

(51) Int. Cl. B29C 55/22; A61M 25/00; B29C 55/00; B29K 627/18; B29L 31/00

(52) U.S. Cl. CPC B29C 55/22; A61M 25/0009; B29C 55/005; B29K 2627/18; B29K 2827/18; B29L 2031/7542

(57) ABSTRACT

A method of manufacturing a fibre comprising a lined channel, using a draw apparatus, the method comprising: providing a preform, comprising a channel extending through the preform, to the draw apparatus; feeding a liner into the channel; heating a portion of the preform; and drawing the heated portion of the preform in order to form a fibre, wherein the liner is held within the channel of the fibre to provide a lined channel within the fibre.

The present application relates generally to a method for forming a fibre comprising a lined channel by way of a fibre drawing technique.

Minimally Invasive Surgery (MIS) procedures, such as interventional radiology and cardiology applications, often require devices with multiple lumens (hollow channels) where other medical devices such as guidewires, balloon catheters, and stent catheters are pushed via the tight confines of these empty lumens. If the internal diameter of these lumens is not of sufficient lubricity, it is possible for devices such as stents to collapse in an accordion-like manner as they are being pushed via the catheter lumen. The effect of increased lubricity of these lumens is a reduced deployment force of devices (guidewire, balloon catheters, stent catheters) as they are passed through the lumen, thereby increasing the probability of a successful procedure.

In order to ensure sufficient lubricity of these lumens, known catheter manufacture methods use melt or non-melt processable fluoropolymer-based liners in the inner walls of catheter lumens to provide smooth inner surfaces. Lubricity is one of many unique characteristics of fluoropolymers that separates them from other polymers. When handling fluoropolymers, their smooth surface and slippery feel are readily apparent. Commonly used fluoropolymers include: PTFE (Polytetrafluoroethylene, coefficient of friction 0.1), FEP (Fluorinated Ethylene-Propylene, coefficient of friction 0.2), PFA (Perfluoroalkoxy, coefficient of friction 0.2), ETFE (Ethylene tetrafluoroethylene, coefficient of friction 0.4), PVDF (Polyvinylidene fluoride, coefficient of friction 0.14-0.17).

To produce devices with properties such as lubricity, known catheter manufacture methods involve extruding fluoropolymer mandrels (no channels), tubes with a single lumen/channel and tubes with multiple lumens/channels. In this case, the extruded bodies will consist of inner channels with the lubricity required for the targeted application. Fluoropolymer mandrels are produced using conventional extrusion techniques. In screw extrusion, polymer melt is pushed through a die of the desired cross-section. To extrude tubing with single/multiple lumens, cross head die extrusion may be used. In cross head die extrusion, the polymer melt enters the die at right angles to the outlet, which allows lumen characteristics to be controlled by individual, pressurized air supplies fed from the back of a cross-head die and into the tube via precision-bore injector needles. For each multi-lumen tubing design, custom tooling are manufactured. Although the majority of polymer extrusion is carried out using the process of screw extrusion, some fluoropolymers cannot be extruded using this method. The polymers which have very low coefficient of friction and/or are difficult to extrude because of their high melt viscosity, are generally ram extruded or non-melt processed instead. In a ram extrusion process, the polymer's granular powder is compacted by a ram and forced through a heated die to give the required extrudate. Unlike screw extrusion, which takes place at high temperatures, ram extrusion takes place at temperatures slightly above room temperature from 40° C. to 60° C.

The method below describes how known catheter manufacture methods use the extruded fluoropolymer liners and mandrels to produce catheters ready for medical use:
(a) An extruded PTFE or stainless steel PTFE-coated mandrel is manually fed through an etched PTFE extruded liner to ensure that the liner doesn't collapse during the process of co-extrusion. Etching alters the surface properties of the polymer allowing it to be bonded to other materials. The PTFE liners are extruded using the cross-head die extrusion technique.
(b) The assembly is then braided with monofilaments (e.g. Liquid Crystal Polymers, Nitinol, Stainless Steel, Kevlar® (Aramid fibres), PEEK) using a micro-braiding machine. Braided reinforcement to the thin wall of plastic tubing improves burst pressure resistance, column strength and torque transmission.
(c) The braided assembly is then manually fed through a polymeric jacket that is light-weight, soft and flexible, with kinking resistance. Commonly used polymers for jacket include: Nylon, Pebax®, a thermoplastic elastomer consisting of rigid polyamide and flexible polyether backbone blocks.
(d) The assembly of step (c) is then manually fed through a peelable (fluoropolymer) heat shrink which is required for reflowing the nylon/Pebax® jacket over braiding to form a finished shaft in catheter design.
(e) The assembly is then heated in a vertically translating hot air nozzle shrinker (thermal traverser) to reflow (fuse) the polymeric jacket to the braided liner.
(f) The peelable heat shrink is peeled off and the mandrel is pulled out of the fused assembly to obtain the single lumen catheter.

Multi-lumen lined tubing may have a second material incorporated into one or more linings of a lumen. For instance, thermoplastic polyurethane may be used as the main multi-lumen body with polyimide (thermoset) liners. The primary benefit of polyimide tubing is its strength-per-unit-size.

In order to produce such multi-lumen lined tubing, it is known to use coextrusion/crosshead die extrusion manufacturing methods. In such methods, the liner mandrel assembly is fed colinearly to the extruder's screw axis. The liner and mandrel assembly are then coated with the polymer melt entering the guide and right angles and once the extrudate is cooled, the mandrel is pulled out.

Known thermal drawing processes start with a preform that is a block of material, often polymer material, from which fibres can be drawn. A preform generally comprises a substantially cylindrical structure, although a preform may comprise any shape.

A longitudinal direction, also referred to herein as the draw direction, can be defined as extending through the length of the preform in the direction along which a fibre would be drawn. A radial direction can be defined as extending radially outwardly from, and perpendicular to, an axis extending in the longitudinal direction (longitudinal axis). An angular velocity will be understood to mean a velocity of rotation of the preform or fibre around the longitudinal axis, resulting in spinning of at least part of the preform as it is drawn into a fibre.

Preforms have a larger transverse cross-sectional area than the fibres which are drawn from them and the resultant fibre which can be drawn from a single preform can be, for example, one thousand times longer than the original preform. During the drawing of a fibre from a preform, the structure shrinks in the transverse direction of the preform and is elongated in the longitudinal direction (the draw direction). As such, the precise structure of the preform in terms of its composition, its shape, its size and any other features has a significant impact on the resulting fibre.

Preforms may be fabricated by one of, or a combination of, a number of techniques including: hot press, cast-moulding or injection moulding of thermoplastic pellets in vacuum; additive manufacturing techniques; direct machining of commercially acquired rods or bars; and rolling of thermoplastic sheets/films and consolidating into preforms.

The process of drawing a fibre comprises heating a portion of the preform to a temperature over its glass transition temperature which in turn allows the neck-down drawing of the preform to take place. During this process, the viscosity of the preform may decrease several orders of magnitude and the preform may be drawn down under its own weight. The drawing temperature should be primarily selected based on the preform material to be used. The higher the glass transition temperature of the preform material to be used, the higher the required draw temperature. It will be appreciated that heating of the preform may be provided by the draw apparatus such as by a resistive heater comprising part of the draw apparatus. Alternatively, heating of the preform may be provided by an external heating apparatus such as a resistive heater, a furnace configured to surround the portion of the preform to be drawn, or any other suitable apparatus. After the drawing of a portion of the preform into a fibre, the resultant fibre may cool (may be quenched) in order to set the shape of the fibre. Cooling of the fibre may occur in ambient conditions as a result of removing the fibre from the influence of the heating apparatus without further need for cooling. Alternatively, the fibre may pass through a region cooled by a cooling apparatus either forming part of the draw apparatus or independently provided. The cooling apparatus may comprise a cooled enclosure through which the drawn fibre passes or may comprise an apparatus for providing cooled air over the fibre. Any suitable method for providing cooling of the fibre may be used. In addition, prior to heating the preform for drawing the fibre therefrom, a pre-heating stage may be provided. Preheating may be performed by any suitable heating apparatus and may be performed in order to avoid thermal shock and to help achieve a better temperature uniformity. It will be appreciated that in some examples, preheating may be unnecessary or may comprise part of the heating step.

The drawing of the fibre may be monitored by a monitoring apparatus. The monitoring apparatus may comprise at least one laser micrometer for measuring the diameter of the fibre or a three-wheel tension sensor for measuring the pulling tension of the fibre by measuring the effective internal stress inside the fibre.

During the drawing of the preform into a fibre, the temperature profile, the down-feed speed and the draw speed may be adjusted. The temperature profile describes the temperature regions through which the preform and resultant fibre pass. The down-feed speed is the rate at which the preform is moved into the heated region. The draw speed is the rate at which the fibre is pulled through the draw apparatus. By varying the temperature profile, the down-feed speed and the draw speed, the thickness of the resultant fibre may be adjusted.

According to an aspect of the invention, there is provided a method of manufacturing a fibre comprising a lined channel, using a draw apparatus, the method comprising: providing a preform, comprising a channel extending through the preform, to the draw apparatus; feeding a liner into the channel; heating a portion of the preform; and drawing the heated portion of the preform in order to form a fibre, wherein the liner is held within the channel of the fibre to provide a lined channel within the fibre.

By means of the present invention, it is possible to manufacture devices with lined channels/lumens by fibre drawing rather than extrusion. In particular, by means of the invention, liners with desired features (lubricity, strength, etc) may be fed into channels formed in a preform that is thermally drawn using a standard drawing apparatus.

In embodiments of the invention, the liner may be etched on its external surface. In such embodiments of the invention, when the liner-mandrel assembly is held by the walls of the channel, the friction between the etched surface of the liner and the channel means that it is difficult, if not impossible to withdraw the liner from the channel.

In embodiments of the invention, the preform may have a larger transverse cross-sectional area than the fibres which are drawn from them and the resultant fibre which may be drawn from a single preform may be, for example, one thousand times longer than the original preform. During the drawing of a fibre from a preform, the structure shrinks in the transverse direction of the preform and is elongated in the longitudinal direction. As such, the precise structure of the preform in terms of its composition, its shape, its size and any other features has a significant impact on the resulting fibre.

The cross-sectional design of the preform may be substantially maintained in the resultant fibre, albeit with smaller transverse dimensions. It is therefore possible to provide a preform with a complex cross-sectional design that is relatively straight forward to produce at a large scale and then draw the preform to form the fibre with the same cross-sectional design at a scale that might otherwise be difficult to produce due to its intricacy.

By means of the present invention it is possible to use existing draw apparatuses to produce a fibre with a lined channel. It may, however, be necessary to modify the existing draw apparatus by retro-fitting a feeder capable of feeding the liner into the channel of the preform.

Hence, at relatively low cost a known draw apparatus may be used to manufacture fibres comprising a lined channel by means of the present invention.

In some embodiments of the invention, the liner may comprise a material with high lubricity characteristics to provide a lined channel that exhibits low internal friction. In other embodiments of the invention, the liner may comprise a material with high strength-per-unit-size to provide a lined channel with greater robustness against devices inserted through it.

Therefore the invention enables manufacture of fibres with enhanced properties such as lubricity and strength that may be particularly advantageous, although not exclusively so, in the field of minimally invasive surgical devices.

In embodiments of the invention the preform further comprises a preform axis and the channel comprises a channel axis.

In such embodiments of the invention the preform axis extends longitudinally through the centre of the preform and the resultant fibre. Similarly, the channel axis extends longitudinally through the centre of the channel.

In embodiments of the invention the channel axis is coaxial with the preform axis.

In such embodiments of the invention the channel therefore extends longitudinally through the centre of the preform and the resultant fibre.

In embodiments of the invention the channel axis is spaced apart from the preform axis.

In such embodiments of the invention the channel is spaced apart from the centre of the preform and resultant fibre. The channel is therefore positioned eccentrically within the cross-section of the preform and resultant fibre.

The channel may extend parallel to the preform axis before the preform is drawn. Further, in some embodiments of the invention, the channel may extend parallel to the preform axis after the preform is drawn into a fibre. However, in other embodiments of the invention, the channel may spiral helically around the preform axis as it extends in the longitudinal direction after the preform is drawn into a fibre.

In embodiments of the invention the step of drawing a heated portion of the preform in order to form a fibre further comprises rotating the preform about the preform axis at an angular velocity such that the resultant fibre comprises a helical lined channel.

In some embodiments of the invention the provision of a helical lined channel rather than a straight lined channel may be advantageous for a number of reasons. For example a liner in a helical lined channel may be better able to adapt to changes in shape of the fibre such as passive bending or twisting. Unlined helical channels introduce more frictional forces when compared to unlined straight channels. Adding liners helps to reduce the high frictional forces experienced while pushing devices through unlined helical channels. Further, if fluid is to flow through the helical lined channels to cool the fibre, the helical channel may provide more efficient cooling as it is more evenly distributed through the volume of the fibre.

The magnitude of the angular velocity of the heated portion of the preform will determine the pitch, i.e., the inter-channel spacing of the coils of a helical channel in the resultant fibre. Where cooling or heating is provided by the circulation of fluid through the helical lined channels, more thorough cooling or heating will be provided in a fibre with a smaller pitch and correspondingly a longer channel path length than for a fibre having a hollow channel with a larger pitch.

The angular velocity may be altered during the drawing process so as to provide different pitches along the length of the resultant fibre. A fibre having different pitches of a helical lined channel therethrough may allow for particularly efficient cooling or heating of areas of the resultant fibre with a small pitch while the remainder of the fibre may not receive much cooling or heating. This may allow for targeted stiffening of segments of the fibre made of polymers with the stiffness tuning property (e.g. shape memory polymers). In one or more examples, the method may include changing one or both of the magnitude and rotational direction of the first angular velocity during the drawing of the first fibre.

Rotation of the preform by 360 degrees may provide for a single loop of a helix to be formed in the resultant fibre. In other embodiments of the invention, the direction of rotation of the preform may be varied during drawing of the preform into a fibre such partially helical channels rather than full helixes are formed. While full helixes may not be formed in the channels of some embodiments of the invention, it will be appreciated that the varying position of a channel, within a fibre's cross-section, along the length of the fibre provides a partially helical structure and that, herein, descriptions of helical channels will encompass such channel shapes.

In embodiments of the invention the step of feeding the liner into the channel comprises feeding the liner from a feeder into a proximal end of the channel, and the method further comprises the step of rotating the feeder around the preform axis at the angular velocity such that the spatial relationship of the feeder relative to the proximal end of the channel remains constant.

In such embodiments of the invention it is possible to feed the liner into the channel continuously while also rotating the preform as it is drawn into a fibre without the liner becoming tangled in the draw apparatus.

In embodiments of the invention the liner comprises a thermoplastic material.

In such embodiments of the invention the thermoplastic material may have high lubricity characteristics. Therefore, by providing a channel lined with a thermoplastic-material liner, the lined channel may exhibit high lubricity characteristics. This may be advantageous if devices, particles or fluids are to be passed through the lined channel. For example, if the fibre is incorporated as part of a medical device, the lined channel may have application as a lumen for deploying a catheter. The increased lubricity provided by the lined channel will reduce the force required to deploy the catheter, reduce the risk of the catheter collapsing and increase the probability of a successful procedure.

In embodiments of the invention the thermoplastic material is a fluoropolymer.

In such embodiments of the invention the fluoropolymer may provide high lubricity characteristics and the lined channel may exhibit increased lubricity when compared to a channel that is not lined.

In other embodiments of the invention the liner may comprise any other suitable thermoplastic material.

In embodiments of the invention the fluoropolymer is polytetrafluoroethylene (PTFE).

In such embodiments of the invention the PTFE liner may have a coefficient of friction of 0.1. In other embodiments of the invention the fluoropolymer may be FEP to provide a coefficient of friction of 0.2, PFA to provide a coefficient of friction 0.2, ETFE to provide a coefficient of friction 0.4 or PVDF to provide a coefficient of friction 0.14-0.17.

In embodiments of the invention the liner comprises a thermoset material.

In such embodiments of the invention the thermoset material may have high strength-per-unit-size. Therefore, by providing a channel lined with a thermoset-material liner, the lined channel may exhibit high strength characteristics. This may be advantageous if devices with hard or sharp edges are to be passed through the lined channel. For example, if the fibre is incorporated as part of a medical device, the lined channel may have application as a lumen for deploying a guidewire. The increased strength provided by the lined channel would reduce the risk of the guidewire damaging the channel and increase the probability of a successful procedure.

In other embodiments of the invention the liner may comprise any suitable material.

In embodiments of the invention the liner has a substantially circular cross-section.

In such embodiments of the invention the liner may be used to line a channel with a circular cross-section. A circular lined channel may be advantageous in applications such as medical devices wherein the channel is used for the deployment of circular devices or for the transport of fluids.

In embodiments of the invention the liner has a non-circular cross-section.

In such embodiments of the invention the liner may be used to line a channel with a non-circular cross-section. A non-circular lined channel may be advantageous in applications wherein correspondingly shaped objects are to be deployed through the channel.

In embodiments of the invention, the method may further comprise the step of feeding a mandrel into the liner, before the liner is fed into the channel, to form a liner-mandrel assembly.

In such embodiments of the invention the step of feeding a liner into the channel may comprise feeding the liner-mandrel assembly into the channel, and the step of drawing the heated portion of the preform in order to form a fibre may result in the liner-mandrel assembly being held within the channel of the fibre.

In such embodiments of the invention, the method may further comprise the step of withdrawing the mandrel from the liner to provide the lined channel within the fibre.

In embodiments of the invention the mandrel comprises a fluoropolymer or a metal coated with a fluoropolymer.

In such embodiments of the invention the fluoropolymer may provide high lubricity characteristics. This means that there will be low friction between the mandrel and the liner, thereby allowing the mandrel to be withdrawn more easily from the liner and with low risk of causing damage to the liner.

In embodiments of the invention the fluoropolymer is polytetrafluoroethylene.

In such embodiments of the invention the PTFE liner may have a coefficient of friction of 0.1. In other embodiments the fluoropolymer may be FEP to provide a coefficient of friction of 0.2, PFA to provide a coefficient of friction 0.2, ETFE to provide a coefficient of friction 0.4 or PVDF to provide a coefficient of friction 0.14-0.17.

In other embodiments the mandrel may comprise any suitable material.

In embodiments of the invention, the method may further comprise the step of removing the liner from the channel in a distal section of the fibre.

In such embodiments of the invention the distal section of the fibre may be more flexible than the rest of the fibre. This may facilitate steering of the fibre during use, such as within tortuous cavities of a human or animal body.

In embodiments of the invention,
the step of providing a preform to the draw apparatus comprises providing a preform, comprising a plurality of channels extending through the preform, to the draw apparatus;
the step of feeding the liner into the channel comprises feeding each of a plurality of liners into a respective one of the plurality of channels; and
the step of drawing the heated portion of the preform in order to form a fibre, provides a plurality of liners held within the respective plurality of channels within the fibre.

In such embodiments of the invention a fibre may be manufactured that comprises a plurality of lined channels.

Each of the plurality of lined channels may comprise a channel axis. In some embodiments the channel axis of one of the plurality of lined channels may be coaxial to the preform axis and therefore extend centrally through the fibre. The channel axes of the other of the plurality lined channels may be spaced apart from the preform axis and therefore extend eccentrically through the fibre. In other embodiments the channel axis of each of the plurality of lined channels may be spaced apart from the preform axis and therefore extend eccentrically through the fibre.

In some embodiments the method of manufacturing the fibre comprising a plurality of lined channels may include steps to manufacture helical lined channels:

The step of drawing a heated portion of the preform in order to form a fibre may further comprise rotating the preform about the preform axis at an angular velocity such that the resultant fibre comprises a plurality of helical lined channels.

The step of feeding each of the plurality of liners into a respective one of the plurality of channels may comprise feeding each of the plurality of liners from a respective one of a plurality of feeders into a proximal end of a respective one of the plurality of channels.

The method may further comprise the step of rotating the plurality of feeders around the preform axis at the angular velocity such that the spatial relationship of each of the plurality of feeders relative to the proximal end of the channel remains constant.

In some embodiments, each of the plurality of liners may comprise the same material to the other liners and, in other embodiments, a liner may comprise a different material to the other liners.

In some embodiments one or more of the liners may comprise a thermoplastic material. In such embodiments of the invention the thermoplastic material may be a fluoropolymer, and in these embodiments, the fluoropolymer may be PTFE.

In some embodiments one or more of the liners may comprise a thermoset material.

In some embodiments the plurality of lined channels may be the same size and shape as each other. In other embodiments, they may have different sizes and/or shapes.

In some embodiments one or more of the liners may comprise a circular cross-section. In some embodiments one or more of the liners may comprise a non-circular cross-section.

In some embodiments each of the plurality of mandrels may comprise a fluoropolymer.

In embodiments of the invention the step of heating a portion of the preform comprises heating a portion of the preform to a temperature less than the melting point of the liner and/or mandrel.

If the liner were to melt during manufacture of the fibre, it is possible that it would be deformed following eventual cooling and solidification. Such deformation may impact the size and shape of the channel within the liner and hence impact the ability for devices, particles or fluids to travel through the channel. Deformation may also result in holes or gaps forming in the liner. This could be especially damaging if the fibre is to be used in applications that require fluid to travel through the lined channel as leakage may occur. Further, melting of the liner may damage or remove etching that might be present on the external surface of the liner. This would negatively affect the ability of the liner to be held within the channel of the fibre.

If the mandrel were to melt and deform it may become difficult or impossible to remove it from the liner. Alternatively, a small portion of the mandrel may become separated from the rest of the mandrel and remain within the liner when the rest of the mandrel is removed. This could result in blockages in the lined channel when the fibre is used.

Hence ensuring that the liner and/or mandrel do not melt during the manufacture process of the fibre is very advantageous.

In embodiments of the invention the method further comprises the step of manufacturing a medical device, wherein the medical device comprises the fibre.

In such embodiments of the invention the dimensions of the fibre may be especially beneficial for a medical device used in minimally invasive surgery applications. The lined channel or plurality of lined channels may be used as: lumens for the deployment of other medical devices such as catheters or guidewires, tendon or pull-wire or cable-receiving channels for the deployment of tendons or pull wires or cables to steer the flexible distal end of a medical device or microfluidic channels for the transport of fluids.

The invention will now be described by way of example only with reference to the accompanying drawings in which:

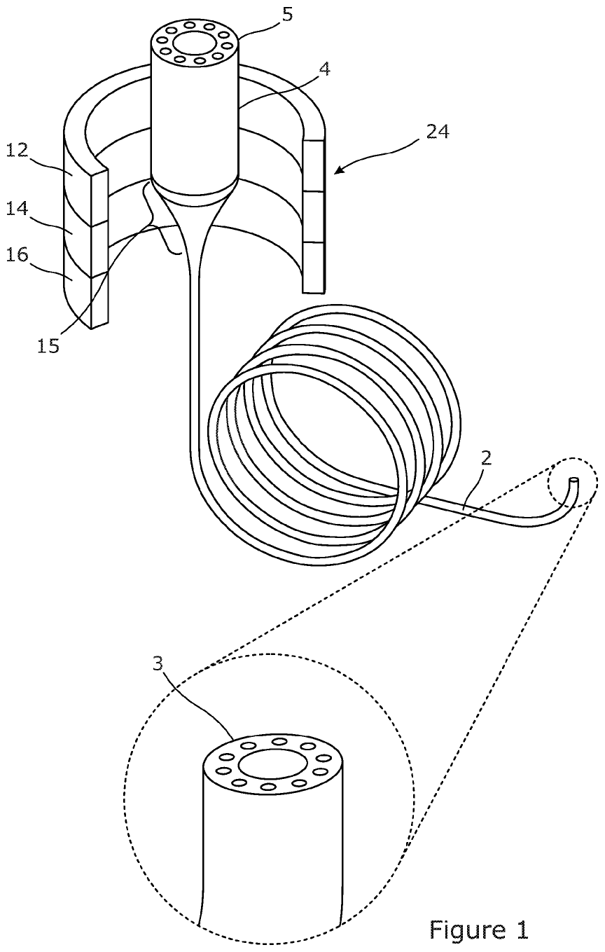

FIG. 1 is a schematic representation of a preform being drawn into a fibre using a known method;

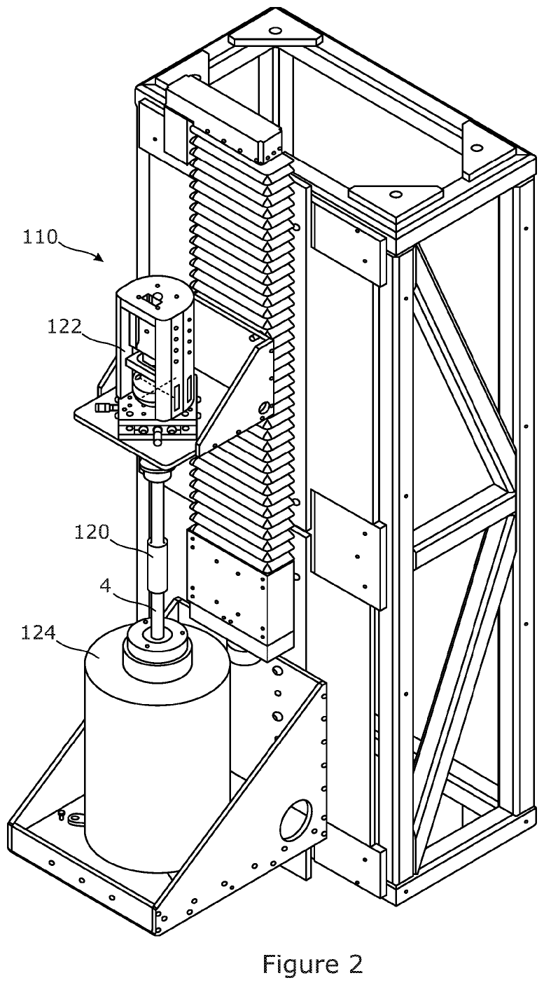

FIG. 2 is an illustration of a known draw apparatus with which a preform may be drawn into a fibre.

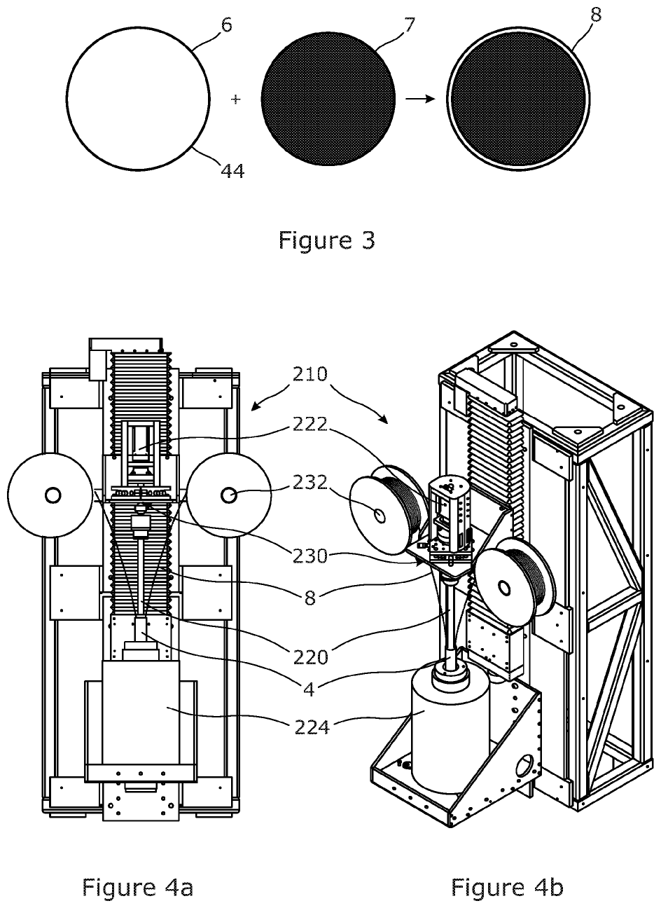

FIG. 3 is a schematic representation of a liner-mandrel assembly formed using a method according to an aspect of the invention;

FIGS. 4a and 4b are illustrations of a draw apparatus for carrying out a method according to an embodiment of the invention.

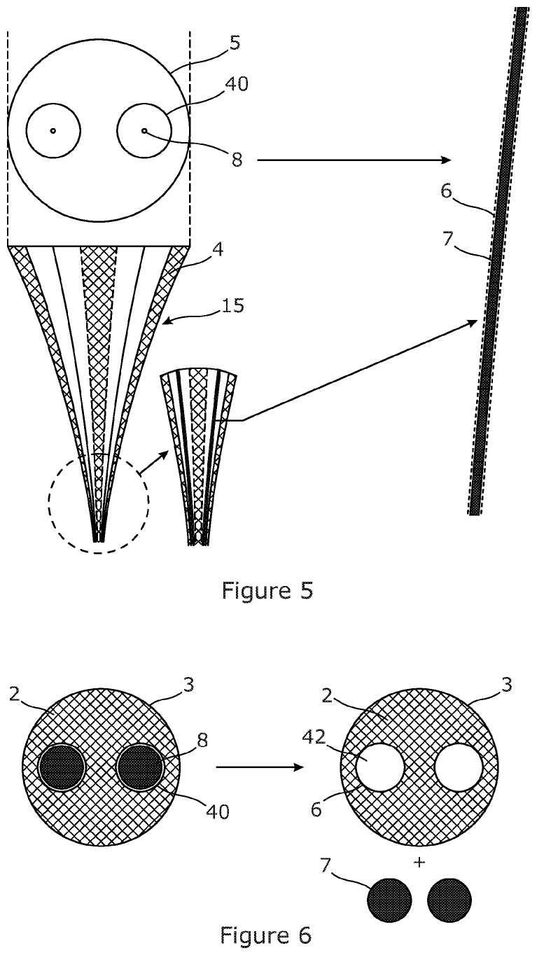

FIG. 5 is a schematic representation of a fibre, comprising a lined channel, being drawn by a method according to an embodiment of the invention.

FIG. 6 is a schematic representation of a lined channel being formed.

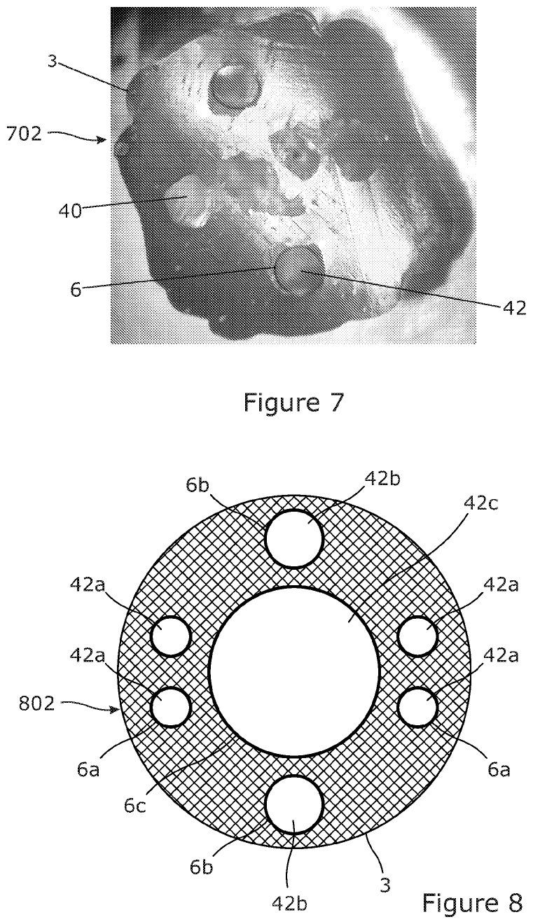

FIG. 7 is a cross-sectional representation of a fibre, comprising a lined channel, manufactured by a method according to an embodiment of the invention.

FIG. 8 is a cross-sectional schematic representation of a fibre, comprising a plurality of lined channels, manufactured by a method according to a further embodiment of the invention.

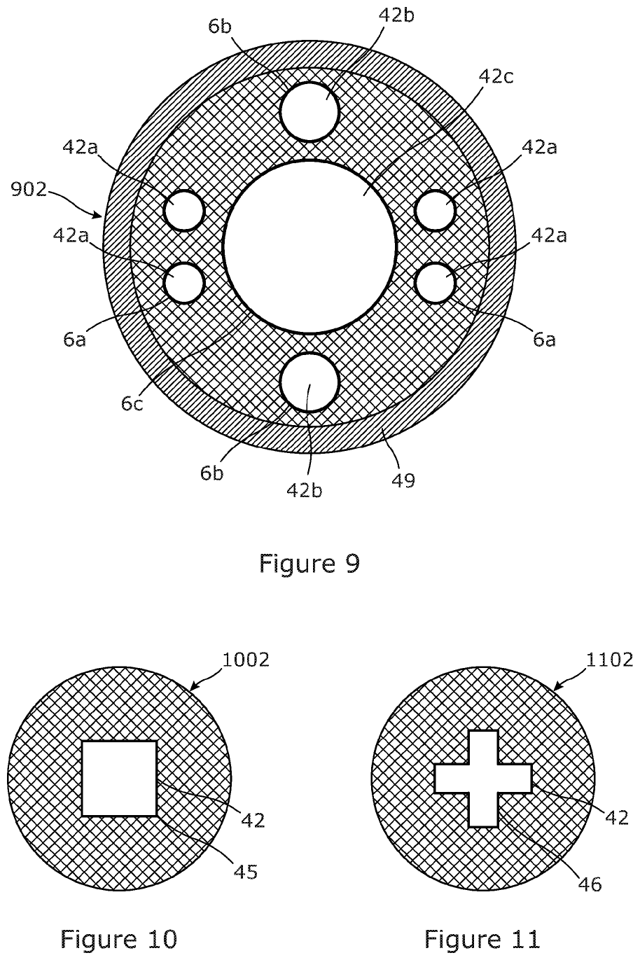

FIG. 9 is a cross-sectional schematic representation of a fibre, comprising a plurality of lined channels, manufactured by a method according to a further embodiment of the invention.

FIG. 10 is a cross-sectional schematic representation of a fibre, comprising a square-shaped lined channel, manufactured by a method according to a further embodiment of the invention.

FIG. 11 is a cross-sectional schematic representation of a fibre, comprising a cross-shaped lined channel, manufactured by a method according to a further embodiment of the invention.

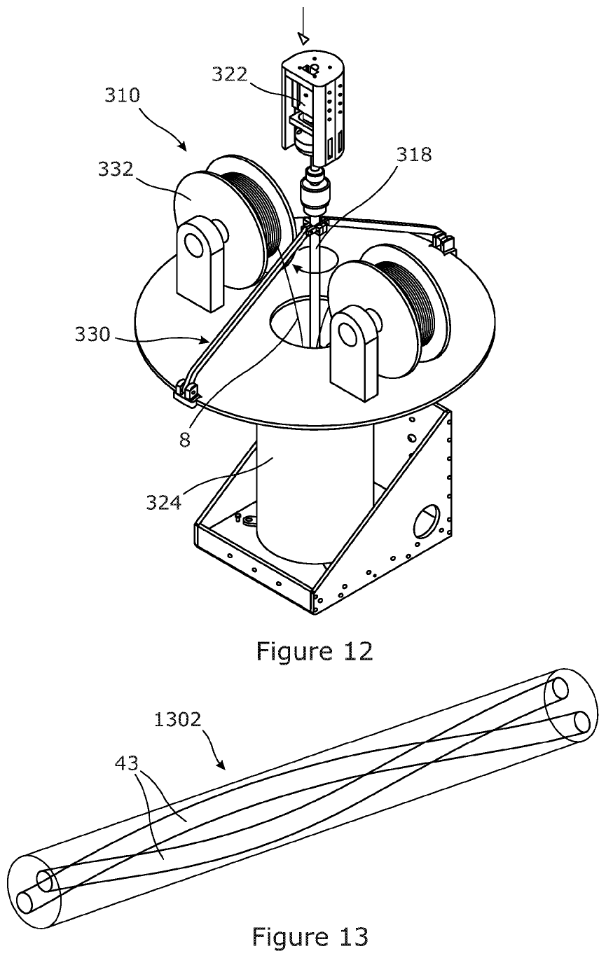

FIG. 12 is an illustration of a draw apparatus for carrying out a method of manufacturing a fibre comprising a helical lined channel similar to that shown in FIG. 4 but according to a different embodiment of the invention.

FIG. 13 is a schematic representation of a fibre comprising a helical lined channel manufactured by the method relating to FIG. 12.

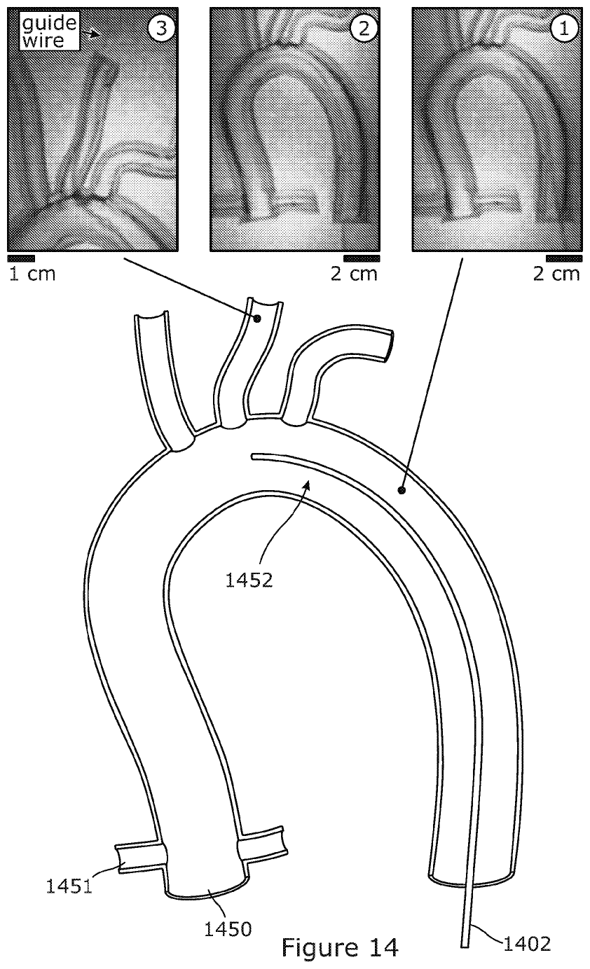

FIG. 14 is a schematic representation of a fibre being fed through an artery during a medical procedure.

Referring initially to FIG. 1, a known method for drawing a fibre is shown wherein the fibre is defined generally by the reference numeral 2. The fibre 2 is drawn from a preform 4 with a preform cross-section 5 wherein the preform is provided to a temperature-controlled apparatus 24 that comprises a pre-heating apparatus 12, a heating apparatus 14 and a quenching apparatus 16. The preform 4 is fed sequentially through the pre-heating apparatus 12 and the heating apparatus 14 in order to raise the temperature of a leading part of the preform 4 and provide a heated portion 15 of the preform 4 that is then suitable to be drawn. The speed of drawing of the fibre 2 may be controlled primarily by gravity or the control of the draw rate may be controlled by a draw apparatus.

In some examples, the preform is initially allowed to neck-down under gravity, after which the tip of the necked-down portion is cut off. Once the necked-down portion has been removed, the remaining drawn fibre may be connected to a capstan which may be used to draw the fibre. Control of the draw speed may be provided by the capstan or may be controlled by any other suitable apparatus. The heated portion 15 of the preform 4 which has been drawn into a fibre 2 is quenched in order to set the fibre shape. Quenching the fibre 2 may be achieved by removing the fibre 2 from the influence of the heating apparatus or, as shown in the example of FIG. 1, the fibre 2 may be cooled to a temperature below the draw temperature by the quenching apparatus 16.

During the drawing process, the design of the preform cross-section 5 is substantially maintained as the preform 4 transitions to the fibre 2 with a fibre cross-section 3. However the dimensions that traverse the fibre cross-section 3 are significantly smaller than those of the preform cross-section 5. It is therefore possible to provide a preform 4 with a complex cross-sectional design that is relatively straight forward to produce at a large scale and then draw the preform 4 to form the fibre 2 with the same cross-sectional design at a scale that might otherwise be difficult to produce due to its intricacy.

Referring now to FIG. 2, a draw apparatus 110 for drawing a fibre using the known method represented in FIG. 1 is shown, wherein the draw apparatus 110 comprises as a draw tower. The draw apparatus 110 comprises a preform mount 120, a preform spinning motor 122 and a temperature-controlled apparatus 124 that is equivalent to the temperature-controlled apparatus 24 shown in FIG. 1.

In this example, a preform 4 is mounted in the draw apparatus 110 by way of the preform mount 120, which may be configured to receive preforms of differing sizes. During drawing of the preform 4 into a fibre, the preform 4 is lowered, by the draw apparatus 110, into the temperature-controlled apparatus 124 in order to provide heating and subsequent cooling of the preform 4 and resultant fibre. When desirable, the preform spinning motor 122 provides rotation of the preform 4 via the preform mount 120 during the draw process.

Providing rotation of the preform 4 during draw may allow for the formation of fibres having helical features therein, such as a helical channel. It will be appreciated that, during drawing of the preform 4 into a fibre, any features in the preform not centrally arranged and radially symmetric in the cross-section of the preform will take on a helical structure or spiralled arrangement in the resulting fibre.

Referring now to FIG. 3, a first step of a method according to an aspect of the invention is shown. A liner 6 is configured to receive a mandrel 7 to form a liner-mandrel assembly 8. In this example, the liner 6 has a circular cross-section 44.

Referring now to FIG. 4, a draw apparatus 210 for drawing a fibre using a method according to an aspect of the invention is shown. The draw apparatus 210 comprises features equivalent to the known draw apparatus 110 of FIG. 2 and further comprises a liner-mandrel feeding apparatus 230 and, in this example, two liner-mandrel spools 232.

In this example, a preform 4 comprising first and second channels extending through the preform may be secured to the draw apparatus via a preform mount 220. First and second liner-mandrel assemblies 8 may then be fed into the first and second channels, respectively, of the preform 4 as it is simultaneously fed into a temperature-controlled apparatus 224. A heated a portion of the preform may then be drawn to form a fibre as is represented in FIG. 5.

Referring now to FIG. 5, a heated portion 15 of a preform 4 is shown being drawn to form a fibre. The preform cross-section 5 shows channels 40, that extend longitudinally through the preform 4, into which liner-mandrel assemblies 8 have been fed. The liner mandrel assemblies 8 have a very small cross-section relative to channels 40 of the preform 4. For example, the channels 40 may have a diameter of 10 mm while the liners may have an outer diameter of 0.5 mm.

However, as the heated portion 15 of the preform 4 is drawn to form a fibre, the preform 4 and the channels within it gradually become narrower in size. The drawing process is continued until a fibre with a desired diameter is formed wherein the diameter of the channel 40 is approximately equal to the outer diameter of the liner, as shown in FIG. 6. The liner 6 may be etched on its external surface so that when the liner-mandrel assembly 8 is surrounded by the walls of the channel 40, there is friction between the etched surface of the liner (6) and the channel (40) making it difficult, if not impossible to withdraw the liner (6) from the channel (40).

Referring now to FIG. 6, a further step of a method according to an aspect of the invention is shown wherein a fibre 2, drawn from a preform as described in relation to FIG. 5, comprises first and second channels 40 that are filled by first and second liner-mandrel assemblies 8 such that the liner mandrel assemblies 8 are held within their respective channels 40. The mandrels 7 of the liner-mandrel assemblies 8 are withdrawn from the liners 6 to form first and second lined channels 42 extending through the length of the fibre 2.

Referring now to FIG. 7, a cross-section 3 of a prototype fibre 702 manufactured by a method according to an aspect of the invention is shown. The fibre 702 was drawn from a 3D printed preform comprising a plurality of channels 40. Two of the channels have been provided as lined channels 42 in accordance with an embodiment of the invention. The liner present in the lined channels 42 may provide beneficial characteristics such as lubricity or strength. Additionally, it can also be seen from FIG. 7 that presence of the liner-mandrel assembly in the channels during the drawing process may improve the degree to which the channels maintain their cross-sectional shape as the fibre is being drawn. The liner channels 42 have generally held their cylindrical shape while the channels 40 that are not lined have deformed more substantially due to reflow deformation driven by surface tension. Such deformation is especially prevalent in preforms/fibres drawn at relatively low viscosities.

Although the method described above uses a mandrel within the liner to help maintain the cross-sectional shape of the channels and liner as the fibre is being drawn, it is not necessary in all cases. For example, depending on the material and/or thickness of the liner, the liner may be sufficiently self-supporting to maintain its shape and that of the surrounding channel. In this scenario the mandrel may be dispensed with. Not only does this simplify the process by removing the steps of feeding the mandrel into the liner before the draw and withdrawing the mandrel from the liner after the draw, but it also reduces the material required to manufacture the fibre and is therefore more cost-effective.

Referring now to FIG. 8, a cross-section 3 of a fibre 802 manufactured by a method according to an embodiment of the invention is schematically represented. The fibre 802 comprises a plurality of lined channels of varying sizes. The channels may have any desired dimensions and features. In this case, there are four small-sized lined channels 42a having diameters in the range 200 to 500 microns, and in this case more specifically 300 microns that may have application as microfluidic channels. There are two medium-sized channels 42b which have diameters in the range 300 to 600 microns and more specifically 450 microns that may have application as tendon or pull-wire or cable receiving channels. Further there is a large-sized channel 42c having a diameter in the range of 800 microns to 1.6 mm, and more specifically 1.4 mm, that may have application as a central lumen. Each of the microfluidic channels 42a, tendon or pull-wire or cable receiving channels 42b and central lumen 42a comprise a correspondingly sized liner 6a, 6b, 6c.

In general, microfluidic channels may have any desired diameter from approximately 100 microns to a few millimetres.

Referring now to FIG. 9, a cross-section 3 of a fibre 902 manufactured by a method according to an embodiment the invention is schematically represented. The fibre 902 comprises all the features of fibre 802 shown in FIG. 8 and further comprises a co-drawn polymer layer 49 that encases the fibre 802.

In some embodiments the co-drawn polymer 49 layer may comprise fluoropolymer to provide the outer surface of the fibre 902 with enhanced lubricity. In other embodiments the co-drawn polymer layer 49 may comprise a thermoset material to provide the fibre 902 with enhanced strength characteristics.

Each of the fibres 802 and 902 shown in FIGS. 8 and 9 respectively may have application being comprised in a medical device in accordance with an embodiment of the invention.

Referring now to FIGS. 10 and 11, two fibres 1002 and 1102 manufactured by methods according to embodiments of the invention are shown that each comprise a single lined channel 42 which extends the length of the respective fibre. In fibre 1002 the lined channel 42 has a square-shaped cross-section 45 and in fibre 1102 the lined channel 42 has a cross-shaped cross-section 46. It will be appreciated that a lined channel may be formed with any suitable shape via methods according to embodiments of the invention.

Referring now to FIG. 12, a draw apparatus 310 for drawing a fibre using a method according to an embodiment of the invention is shown. The draw apparatus 310 comprises equivalent features to the draw apparatus 210 of FIG. 4 wherein a liner-mandrel feeding apparatus 330 is coupled to a preform mount 320 that may be rotated by a preform spinning motor 322. This enables the liner-mandrel feeding apparatus 330 to be rotated with the same angular velocity as the preform, thereby apparatus that each liner-mandrel spool 332 remains in-line with a channel in the preform into which the liner-mandrel assembly 8 is being fed. Further, this protects the liner-mandrel assembly 8 from becoming tangled in the draw apparatus 310 as it is being fed into a rotating preform.

By rotating the preform as it is being drawn to form a fibre while also maintaining alignment of the liner-mandrel feeding apparatus 330, a fibre 1302 comprising a helical lined channel 43 can be manufactured using a method according to an embodiment of the invention. An example of the resulting fibre 1302, comprising helical lined channels 43, is shown in FIG. 13.

If the preform that was drawn to form either of the two fibres 1002 and 1102 was rotated during the drawing process, similarly to the method described in relation to FIG. 12, then it would be possible to create spiralled square and cross-shaped channels respectively wherein the cross-sectional shape of the channel spirals as it extends through the fibre. It will be appreciated that a lined channel with any suitable cross-sectional shape may be spiralled in this way.

FIG. 14 shows one possible application of a fibre 1402 described herein. In this example, the fibre 1402 is being fed through a model of an artery 1450 and connected blood vessels 1451 during a mock medical procedure. The lined channel of the fibre 1402 may facilitate the insertion of an endoscope for imaging the internal structure of the artery 1450 or blood vessels 1451 or of a tool for removing a build-up of plaque from the inner walls of the artery 1450 or blood vessels 1451. Furthermore, by removing the liner from the channel in a distal section 1452 of the fibre 1402, it may be easier for a clinician to steer the fibre 1402 through the artery 1450 and blood vessels 1451. Removal of the liner from the distal section 1452 may be performed mechanically (e.g. by cutting or abrasion), optically (e.g. using a laser) or chemically (e.g. via a selective wet chemical etch). The inset images 1-3 were captured at different stages of the procedure. Inset 1 shows the fibre 1402 approaching the bend in the artery 1450 whilst inset 2 shows the fibre 1402 after having navigated the bend. Inset 3 shows a guide wire being used to help direct the fibre 1402 into one of the connected blood vessels 1451.

1. A method of manufacturing a fibre comprising a lined channel, using a draw apparatus, the method comprising:
providing a preform, comprising a channel extending through the preform, to the draw apparatus;
feeding a liner into the channel;
heating a portion of the preform; and
drawing the heated portion of the preform in order to form a fibre, wherein the liner is held within the channel of the fibre to provide a lined channel within the fibre.

2. The method of claim 1, wherein the preform further comprises a preform axis and the channel comprises a channel axis.

3. The method of claim 2, wherein the channel axis is coaxial with the preform axis.

4. The method of claim 2, wherein the channel axis is spaced apart from the preform axis.

5. The method of claim 4, wherein the step of drawing a heated portion of the preform in order to form a fibre further comprises rotating the preform about the preform axis at an angular velocity such that the resultant fibre comprises a helical lined channel.

6. The method of claim 5, wherein the step of feeding the liner into the channel comprises feeding the liner from a feeder into a proximal end of the channel, and the method further comprises the step of rotating the feeder around the preform axis at the angular velocity such that the spatial relationship of the feeder relative to the proximal end of the channel remains constant.

7. The method of claim 1, wherein the liner comprises a thermoplastic material.

8. The method of claim 7, wherein the thermoplastic material is a fluoropolymer.

9. The method of claim 8, wherein the fluoropolymer is polytetrafluoroethylene.

10. The method of claim 1, wherein the liner comprises a thermoset material.

11. The method of claim 1, wherein the liner has a substantially circular cross-section, or the liner has a non-circular cross-section.

12. The method of claim 1, further comprising the step of feeding a mandrel into the liner, before the liner is fed into the channel, to form a liner-mandrel assembly, wherein:
the step of feeding a liner into the channel comprises feeding the liner-mandrel assembly into the channel; and
the step of drawing the heated portion of the preform in order to form a fibre results in the liner-mandrel assembly being held within the channel of the fibre.

13. The method of claim 12, further comprising the step of withdrawing the mandrel from the liner to provide the lined channel within the fibre.

14. The method of claim 12, wherein the mandrel comprises a fluoropolymer, optionally the fluoropolymer is polytetrafluorocthylene.

15. The method of claim 1, further comprising the step of removing the liner from the channel in a distal section of the fibre.

16. The method of claim 1, wherein:
the step of providing a preform to the draw apparatus comprises providing a preform, comprising a plurality of channels extending through the preform, to the draw apparatus;
the step of feeding the liner into the channel comprises feeding each of a plurality of liners into a respective one of the plurality of channels; and
the step of drawing the heated portion of the preform in order to form a fibre, provides a plurality of liners held within the respective plurality of channels within the fibre.

17. The method of claim 1, wherein the step of heating a portion of the preform comprises heating a portion of the preform to a temperature less than the melting point of the liner.

18. The method of claim 1, further comprising the step of manufacturing a medical device, wherein the medical device comprises the fibre.
